(19) United States
(12) Patent Application Publication
(10) Pub. No.: US 2025/0388864 A1
(43) Pub. Date: Dec. 25, 2025

(54) ANIMAL ORIGIN-FREE CELL SHEET, PREPARATION METHOD THEREFOR AND USE THEREOF

(71) Applicants: BOE REGENERATIVE MEDICINE TECHNOLOGY CO., LTD., Beijing (CN); BOE TECHNOLOGY GROUP CO., LTD., Beijing (CN)

(72) Inventors: Dehua CHANG, Beijing (CN); Xin JIN, Beijing (CN); Shuang GAO, Beijing (CN); Juan WANG, Beijing (CN); Shuai LIU, Beijing (CN); Yufei ZHAO, Beijing (CN)

(21) Appl. No.: 18/842,631

(22) PCT Filed: Apr. 27, 2023
(86) PCT No.: PCT/CN2023/091164

§ 371 Date: Apr. 4, 2025

(30) Foreign Application Priority Data
Apr. 29, 2022 (CN) 202210475292.9

(51) Int. Cl. C12N 5/0775

(52) U.S. Cl. CPC C12N 5/0665; C12N 2500/30; C12N 2501/10; C12N 2513/00; C12N 2533/32; C12N 2533/50; C12N 2533/52; C12N 2533/54

(57) ABSTRACT

The present disclosure provides an animal origin-free (for example, serum-free) cell sheet, such as a mesenchymal stem cell sheet, and a preparation method for the animal origin-free cell sheet. The present disclosure further provides use of the animal origin-free (e.g., serum-free) cell sheet, such as the mesenchymal stem cell sheet, in damage repair of damaged tissue.

### TECHNICAL FIELD

The present disclosure relates to the fields of tissue engineering and regenerative medicine, and in particular to animal origin-free cell sheets and preparation methods and use thereof.

### BACKGROUND OF THE INVENTION

Cell sheets represent a more efficient method of stem cell transplantation than previous transplantation methods involving the injection of cells with stem cell suspensions. Cell sheets can effectively prevent the loss of stem cells during the transplantation process and improve the efficiency of stem cell transplantation. In addition, the preparation process of cell sheets does not involve using of enzymes and analogues for cell digestion, which effectively avoids the destruction of the extracellular matrix and reduction of cell functions caused by enzyme digestion. Cell sheet transplantation allows stem cells to better perform their functions in vivo.

The preparation process of mesenchymal stem cell sheets usually involves digesting the subcultured mesenchymal stem cells into single cells with biological enzymes, followed by washing to remove the residue of mesenchymal stem cell culture medium, then resuspending the cells in sheet-forming medium, and inoculating the cells into a pre-coated temperature-sensitive culture dish. Under the culture conditions of 37° C., the cells grew adherently in the culture dish and proliferated to confluence. After the culture is completed, the culture temperature is lowered, and the cells will automatically detach from the temperature-sensitive surface in the form of sheets, and the cell sheets can be harvested.

Cell sheets are film-like structures composed of a single layer or multiple layers of cells. The sheet-forming medium and the matrix coated in temperature-sensitive culture dishes used in the preparation of mesenchymal stem cell sheets remain in the intercellular spaces and on the surface, and are difficult to be completely washed away by external force. The sheet-forming medium used in the sheet preparation methods that have been reported so far is divided into three categories: serum-containing medium, commercially available serum-free medium, and self-prepared serum-free medium. In the traditional serum sheet-forming system, serum can promote the attachment of cells to the surface of temperature-sensitive culture dishes and promote adhesion between cells. However, the bovine serum in the serum-containing medium contains bovine serum albumin, a heterologous macromolecule allergic component, which is not safe enough for clinical use. Due to the lack of serum in the serum-free culture system, it is difficult for the cells to adhere to the wall, and are unable to form a sheet but appear in a broken state, or the sheet is easily broken due to insufficient toughness during the sheet obtaining process (FIG. 1). Commercially available serum-free medium for mesenchymal stem cells and self-prepared serum-free medium for mesenchymal stem cells in the literature contain a variety of exogenous growth factors in order to support cell growth, and the residues of exogenous growth factors can also pose safety risks.

In addition, the current coating matrix in the sheet preparation process is scientific research grade reagents such as fibronectin and laminin. These reagents do not meet GMP standards during the production process.

Therefore, there is an urgent need to develop a method for preparing mesenchymal stem cell sheets with good shape and toughness using a safer sheet-forming medium and/or safer coating matrixes, so as to obtain a mesenchymal stem cell sheet that is safer, easier to store and use.

### SUMMARY OF THE INVENTION

In order to solve the above technical problems, the present disclosure provides a production method for mesenchymal stem cell sheet products. The sheet-forming medium used in this method has simple ingredients, including a basal medium and a binder (such as human serum albumin (such as pharmaceutical grade)), which does not contain any animal-derived components (such as serum) and exogenous growth factors. In addition, during the preparation of cell sheets, adhesion factors (such as human fibrinogen) can be used as a coating matrix.

The mesenchymal stem cell sheet product prepared by the above method of the present disclosure is safer and does not contain animal-derived components (such as serum) and exogenous growth factor residues (such as bovine serum albumin residues that meet drug standards), and the medium formula used in the sheet formation process is simple. In addition, the mesenchymal stem cell sheet product of the present disclosure has a certain degree of toughness and can be folded and flattened in appropriate culture medium, buffers or preservation solutions. The sheet products can still maintain good sheet shape after being stored in a specific preservation solution at 4° C. for 24 hours, with the cell survival rate as high as over 70%; the cells can secrete high levels of pro-angiogenic factors and anti-inflammatory factors, inhibit lymphocyte proliferation and inflammatory factor secretion, and can be used clinically for a variety of diseases, including autoimmune system diseases, organ damage diseases, rejection and GVHD during organ transplantation, etc.; at the same time, the cells can maintain stem cell properties, have specific surface markers, and have the ability of three-direction differentiation when induced, and can be used clinically to repair damaged tissues.

Accordingly, the present disclosure relates to the following aspects.

In the first aspect, the present disclosure relates to a method of preparing a cell sheet, the method comprising the following steps:
a. cultivating and passaging cells;
b. transferring the cells to a temperature-sensitive culture dish coated with a matrix (also known as adhesion factor) and culturing the cells in a sheet-forming medium to form a sheet in the culture dish, wherein the sheet-forming culture contains a basal culture medium and a binder (such as human serum albumin) and does not contain animal-derived components (such as serum, e.g., bovine serum albumin, etc.) and exogenous growth factors; and
c. detaching the cells from the temperature-sensitive culture dish by lowering the temperature.

The term “temperature-sensitive culture dish” or “thermosensitive culture dish” as used herein refers to a culture dish whose surface is coated with a layer of temperature-sensitive polymer material. The stretching states of the molecular chain segments of the polymer material are different at different temperatures to show hydrophilicity or hydrophobicity, so that the hydrophilicity and hydrophobicity of the polymer material can change with changes in external temperature. When the surface of the temperature-sensitive culture dish becomes hydrophilic, the adhesion to the cells and the extracellular matrix secreted by the cells become poor, and the cells will fall off in layers. In a specific application, when the temperature is lowered below the low critical dissolution temperature of the polymer substance, the surface of the temperature-sensitive culture dish becomes hydrophilic, so that the cells will fall off in layers.

In some embodiments, the cell sheet is a stem cell sheet, such as a mesenchymal stem cell sheet.

In some embodiments, the matrix is fibronectin, laminin, gelatin, collagen, vitronectin, or human fibrinogen. In some embodiments, the matrix is gelatin. In some embodiments, the concentration of gelatin (w/w) is 0.01-0.5%, such as 0.05-0.2%, such as 0.1%. In some embodiments, the matrix is vitronectin. In some embodiments, the concentration of vitronectin is 1-20 μg/mL, such as 5-15 μg/mL, such as 10 μg/mL. In some embodiments, the matrix is poly-D-lysine (PDL). In some embodiments, the concentration of PDL is 0.01-0.5 mg/mL, such as 0.05-0.2 mg/mL, such as 0.1 mg/mL.

Coating a temperature-sensitive culture dish with the above matrix enables mesenchymal stem cells to attach to the culture dish.

In a preferred embodiment, the matrix is human fibrinogen. In some embodiments, the concentration of human fibrinogen is 0.1-10 mg/mL, such as 0.2-5 mg/mL, such as 1-2.5 mg/mL.

In some embodiments, in step b, the basal medium in the sheet-forming medium can be selected from the group consisting of DMEM (high glucose), DMEM (low glucose), RPMI1640, α-MEM, DMEM/F12, and F12. In a preferred embodiment, the basal medium in the sheet-forming medium is α-MEM.

In some embodiments, in step b, the concentration of human serum albumin in the sheet-forming medium is 0.1-10%, preferably 0.1-5%, such as 0.1%, 0.5%, 1%, 1.5%, 2%, 2.5%, 3%, 3.5%, 4%, 4.5% or 5%.

In some embodiments, in step b, the sheet-forming medium further comprises non-essential amino acids (glycine, L-alanine, L-aspartic acid, L-asparagine, L-glutamic acid, L-proline, L-serine) and/or L-glutamine.

In some embodiments, the concentration of L-glutamine in the sheet-forming medium used in step b is 0.5 mM to 4 mM, preferably about 2 mM.

In some embodiments, the concentration of each of the non-essential amino acids glycine, L-alanine, L-aspartic acid, L-asparagine, L-glutamic acid, L-proline and L-serine is 50 μM to 200 μM, preferably about 100 μM.

In some embodiments, in step a, the mesenchymal stem cells are cultured and passaged with a serum-containing medium (such as a medium containing fetal bovine serum).

In some embodiments, in step a, the mesenchymal stem cells are cultured and passaged using a serum-free medium. In some embodiments, the serum-free culture system includes the use of a basal medium selected from the group consisting of: RPMI1640, DMEM, α-MEM, DMEM/F12, and F12 serum-free medium, and the medium is supplemented with one or more additives selected from the group consisting of: vitamin C, sodium selenate, hydrocortisone, insulin, transferrin, human serum albumin (plant expression), progesterone, putrescine, biotin, sodium pyruvate, ethanolamine, carnitine, amino acids, vitamins, glutathione, linoleic acid and linolenic acid.

In other embodiments, the serum-free culture system includes use of a commercial medium selected from: CTS™ Stem Pro™ MSC SFM, MesenCult™-ACF Medium, MesenCult™-ACF Plus Medium, and MesenCult™-XF medium.

In some embodiments, the method further includes a step of washing the cells after step a and before step b.

In some embodiments of the above methods, the mesenchymal stem cells can be derived from a tissue selected from the group consisting of: amniotic fluid, amnion, chorion, chorionic villi, decidua, placenta, umbilical cord blood, umbilical cord, adult bone marrow, adult peripheral blood and adult adipose tissue.

In some embodiments, the mesenchymal stem cells can be selected from umbilical cord mesenchymal stem cells, placental mesenchymal stem cells, adipose mesenchymal stem cells, and bone marrow mesenchymal stem cells, as well as mesenchymal stem cells from other sources known in the art. In a preferred embodiment, the mesenchymal stem cells are umbilical cord mesenchymal stem cells.

In some embodiments, the mesenchymal stem cells are umbilical cord mesenchymal stem cells, and the method further comprises a step of obtaining mesenchymal stem cells from the umbilical cord before step a).

In some embodiments, obtaining umbilical cord mesenchymal stem cells from the umbilical cord comprises the following steps:
i. separating a Wharton's jelly from the umbilical cord;
ii. cutting the Wharton's jelly into small tissue pieces, and culturing the tissue pieces in a serum-free culture system for a sufficient time to allow mesenchymal stem cells to migrate from the tissue pieces; and
iii. removing the tissue pieces to obtain the umbilical cord mesenchymal stem cells when the mesenchymal stem cells grow to 50%-100% confluence, such as 70%-100% confluence or 80%-100% confluence.

In some embodiments, the coating time of the temperature-sensitive culture dish is from 1 hour to 7 days, preferably from 1 to 36 hours, and most preferably from 2 to 18 hours. In some embodiments, the coating temperature is 2-37° C., preferably 37° C.

In some embodiments, the cells are inoculated into a temperature-sensitive culture dish in an inoculation amount as shown below.

When inoculated into a 100 mm culture dish, the number of inoculated cells is 1×10⁷ to 10×10⁷, such as 1×10⁷, 2×10⁷, 3×10⁷, 4×10⁷, 5×10⁷, 6×10⁷, 7×10⁷, 8×10⁷, 9×10⁷ or 10×10⁷. The volume of the culture medium in which the inoculated cells are suspended is 15-30 mL, such as 15, 20, 25 or 30 mL.

When inoculated into a 35 mm culture dish, the number of inoculated cells is 1×10⁶ to 30×10⁶, such as 2×10⁶ to 20×10⁶, 5×10⁶ to 15×10⁶, or 8×10⁶ to 12×10⁶, such as 8×10⁶, 9×10⁶, 10×10⁶, 11×10⁶ or 12×10⁶. The volume of the culture medium in which the inoculated cells are suspended is 1.5-5 mL, such as 1.5, 2, 2.5, 3, 3.5, 4, 4.5 or 5 mL.

When inoculated into a 60 mm culture dish, the number of inoculated cells is 3×10⁶ to 75×10⁶, such as 5×10⁶ to 60×10⁶, 10×10⁶ to 50×10⁶, 15×10⁶ to 40×10⁶, 20×10⁶ to 30×10⁶ or 20×10⁶ to 25×10⁶, such as 20×10⁶, 20.5×10⁶, 21×10⁶, 21.5×10⁶, 22×10⁶, 22.5×10⁶, 23×10⁶, 23.5×10⁶, 24×10⁶, 24.5×10⁶ or 25×10⁶. The volume of culture medium in which the inoculated cells are suspended is 3-12.5 mL, such as 3, 3.5, 4, 4.5, 5, 5.5, 6, 6, 5, 7, 7.5, 8, 8.5, 9, 9.5, 10, 10.5, 11, 11.5, 12 or 12.5 mL.

In the method of the present disclosure, the mesenchymal stem cells are detached from the temperature-sensitive culture dish by lowering the temperature, thereby forming a mesenchymal stem cell sheet. For example, when the culture temperature is about 37° C., the mesenchymal stem cells are detached from the temperature-sensitive culture dish by lowering the temperature to 4-32° C.

In a second aspect, the present disclosure relates to a cell sheet prepared by the methods of the present disclosure.

The residual amount of bovine serum albumin in the cell sheet obtained by the above preparation method complies with the human drug standard, and the content is ≤1 ng/cm², the residual amount of human serum albumin is 10 ng/cm²-15000 ng/cm², and the residual amount of human fibrinogen is 0.5 ng/cm²-5 ng/cm².

The mesenchymal cell sheet obtained by the above preparation method can secrete a large amount of HGF, VEGF and IL-8 pro-angiogenic function-related factors, secrete a large amount of anti-inflammatory related factors such as IL-6 and IL-8; and has the function to inhibit lymphocyte Th1 subtype as well as inhibit lymphocyte proliferation and lymphocyte TNFα secretion.

In a third aspect, the present disclosure relates to the use of the mesenchymal stem cell sheet obtained by the above preparation method for regulating an inflammatory response or treating an autoimmune system disease in a subject, or the use in the manufacture of a medicament for regulating inflammatory response or treating autoimmune system diseases.

In some embodiments, the autoimmune disease is rheumatoid, allergy, lupus erythematosus, etc.

In a fourth aspect, the present disclosure relates to the use of the mesenchymal stem cell sheet obtained by the above preparation method for damage repair of damaged tissue in a subject, or the use in the manufacture of a medicament for repairing damaged tissue in a subject.

In some embodiments, the damaged tissue is a tissue of heart, liver, pancreas, uterus, or another tissue.

### DESCRIPTION OF THE DRAWINGS

FIG. 1 shows the effect of serum as a coating matrix on sheet formation of the cell sheets. FBS: fetal bovine serum.

FIG. 2 shows an exemplary appearance of the cell sheets.

FIG. 3 shows the effect of the presence or absence of human fibrinogen as a coating matrix on sheet formation of the cell sheets in a serum-free system.

FIG. 4 shows the effect of the presence or absence of human serum albumin as a binder in the sheet-forming medium on sheet formation of the cell sheets in a serum-free system.

FIG. 5 shows the results of IHC staining of the cell sheets.

FIG. 6 shows the results of H&E staining of the cell sheets.

FIG. 7 shows photos of the cell sheets from different batches. Samples 1-3 are independent sheet samples.

FIG. 8 shows the results of the residual amount of bovine serum albumin in the cell sheets. Samples 1-3 are independent sheet samples.

FIG. 9 shows the results of the residual amount of human serum albumin in the cell sheets. Samples 1-3 are independent sheet samples.

FIG. 10 shows the results of the residual amount of human fibrinogen in the cell sheets. Samples 1-3 are independent sheet samples.

FIG. 11 shows the secretion of HGF in cell culture medium, sheet culture medium, and sheet re-attachment culture medium. The results showed that the secretion of HGF was detected in all three media, and the secretion amount of HGF in the sheet culture medium and sheet re-attachment culture medium was significantly higher than that in the cell culture medium. Samples 1-3 are independent sheet samples.

FIG. 12 shows the secretion of VEGF in cell culture medium, sheet culture medium, and sheet re-attachment culture medium. The results showed that the secretion of VEGF was detected in all three media, and the amount of VEGF secreted in the sheet re-attachment culture medium was much higher than that in the cell culture medium and the sheet culture medium. Samples 1-3 are independent sheet samples.

FIG. 13 shows the secretion of IL-6 in cell culture medium, sheet culture medium, and sheet re-attachment culture medium. The results showed that the secretion of IL-6 in the sheet culture medium and sheet re-attachment culture medium was significantly higher than that in the cell culture medium. Samples 1-3 are independent sheet samples.

FIG. 14 shows the secretion of IL-8 in cell culture medium, sheet culture medium, and sheet re-attachment culture medium. The results showed that the secretion of IL-8 in the sheet culture medium and sheet re-attachment culture medium was significantly higher than that in the cell culture medium. Samples 1-3 are independent sheet samples.

FIG. 15 shows the inhibitory effect of the sheets on the lymphocyte subpopulation Th1. The results showed that sample 1 had an inhibitory rate of 23.5% for lymphocyte subpopulation Th1, sample 2 had an inhibitory rate of 16.3% for lymphocyte subpopulation Th1, and sample 3 had an inhibitory rate of 20.1% for lymphocyte subpopulation Th1. Samples 1-3 are independent sheet samples.

FIGS. 16A-16B show the inhibitory effect of the sheets on lymphocyte proliferation. FIG. 16A shows that sample 1 inhibits lymphocyte proliferation by 56.2%, sample 2 inhibits lymphocyte proliferation by 57.8%, and sample 3 inhibits lymphocyte proliferation by 67.6%. FIG. 16B shows that mesenchymal stem cells in the cell sheets have a significant inhibitory effect on mononuclear cells. Samples 1-3 are independent sheet samples.

FIG. 17 shows the inhibitory effect of the sheets on TNFα secretion by lymphocytes. The results show that the sheets have an inhibitory effect on the secretion of TNFα by lymphocytes in an inflammatory environment, indicating that the sheets have immunomodulatory and anti-inflammatory effects. Samples 1-3 are independent sheet samples.

FIGS. 18A-18C show the results of flow cytometry of surface markers on the cell sheets of Sample 1, Sample 2, and Sample 3.

FIGS. 19A-19C show the results of three-direction differentiation by induction of mesenchymal stem cells in the cell sheets of Sample 1, Sample 2, and Sample 3. FIG. 19A shows the results of adipogenic differentiation of mesenchymal stem cells in the cell sheets, FIG. 19B shows the results of osteogenic differentiation of mesenchymal stem cells in the cell sheets, and FIG. 19C shows the results of chondrogenic differentiation of mesenchymal stem cells in the cell sheets.

FIGS. 20A-20D show photos of the cell sheets prepared in a serum-free sheet-forming system using different coating matrixes and a sheet-forming medium containing HSA. FIG. 20A shows photos of the cell sheets prepared using gelatin as the coating matrix. FIG. 20B shows photos of the cell sheets prepared using vitronectin XF as the coating matrix. FIG. 20C shows photos of the cell sheets prepared using PDL as the coating matrix. FIG. 20D shows photos of the cell sheets prepared using recombinant fibronectin as the coating matrix.

### DETAILED DESCRIPTION OF THE INVENTION

The invention is further illustrated by the following examples, but any example or combination thereof should not be construed as limiting the scope or implementation of the invention. The scope of the present invention is defined by the appended claims. A person of ordinary skill in the art can clearly understand the scope defined by the claims based on this description and common knowledge in the art. Without departing from the spirit and scope of the present invention, those skilled in the art can make any modifications or changes to the technical solution of the present invention, and such modifications and changes are also included in the scope of the present invention.

### Example 1. Isolation and Culture of Umbilical Cord Mesenchymal Stem Cells

The collected newborn umbilical cord was washed with physiological solution, the arteries, veins, and adventitia were removed, and the Wharton's jelly was isolated, cut into small tissue pieces of 0.1˜2 mm, and the tissue pieces were plated evenly into a culture container coated with matrix, with the distance between tissue blocks being 2˜30 mm. Then the culture container was placed in a cell culture incubator. After 2 to 7 days, an appropriate amount of complete culture medium (α-MEM supplemented with 20 IU/mL bFGF and 10% fetal bovine serum) was added to cover the tissue block. The umbilical cord mesenchymal stem cells migrated out can be observed after 8 to 21 days.

When the cells were to 70˜100% confluence, the tissue pieces were removed and the cells were subcultured. The cells were separated from the culture container by trypsinization and cell scraping. Then, the cells were dispersed in the culture medium by stirring, vortexing, etc., and the cells were seeded into a culture container at a density of 500 to 100,000 cells/cm². An appropriate amount of α-MEM supplemented with 20 IU/mL bFGF and 10% fetal bovine serum was added. The culture medium was replaced with an appropriate amount of fresh culture medium every 1 to 5 days according to the cell growth status. When the cells were expanded to 70 to 100% confluence, cell passage was repeated. The cultured umbilical cord mesenchymal stem cells grew adherently in a fibrous form with uniform morphology.

### Example 2. Preparation of Umbilical Cord Mesenchymal Stem Cell Sheets

Before preparing the umbilical cord mesenchymal stem cell sheets, 0.1-5 mg/mL human fibrinogen was firstly used to coat the temperature-sensitive culture dish, which helps the mesenchymal stem cells adhere to the inner surface of the culture dish. The temperature-sensitive culture dish was coated at 37° C. for 2 hours, and then the coating solution was discarded.

The old culture medium was removed from the umbilical cord mesenchymal stem cell culture medium obtained in Example 1, then the cells were washed with PBS for 1-3 times, and the digestive enzyme TryPLE (purchased from Life technologies, Catalog number 12604021) was added for digestion until the cells were in the single-cell state. PBS was added to stop or reduce the digestion of the enzyme. After centrifugation, the supernatant was discarded to obtain a cell pellet.

The digested cells were washed with PBS buffer for 3 times at a washing density of less than or equal to 5×10\hat{ }5 cells/mL.

The cells were washed to remove the residue of umbilical cord mesenchymal stem cell culture medium, and then sheet-forming medium (α-MEM, containing 50 μM to 200 μM non-essential amino acids, 0.5 mM to 4 mM L-glutamine, 0.1%-10% human serum albumin) was added to resuspend the cells, then the cells were preheated to 37° C. and inoculated in a temperature-sensitive culture dish that has been coated with a matrix facilitating cell adhesion. The cells grew adherently in the culture dish.

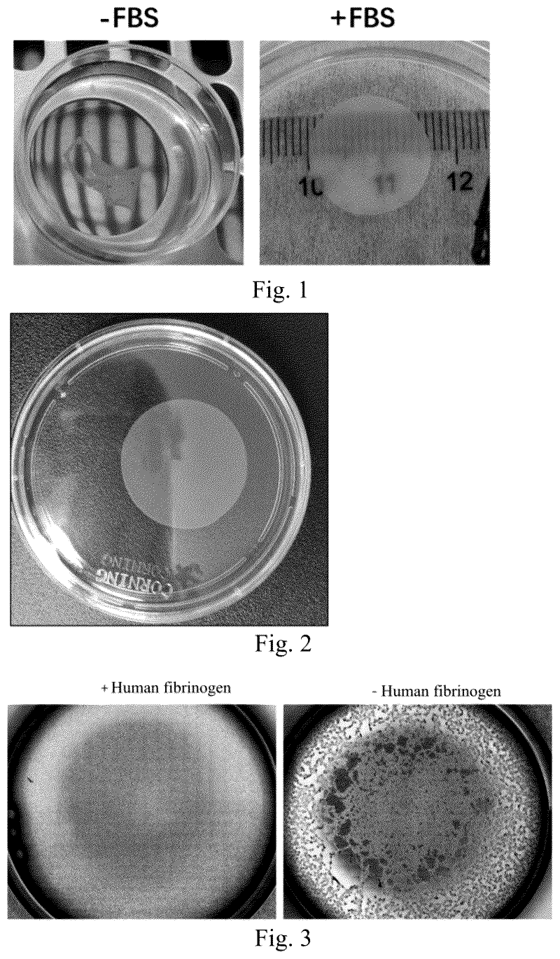

When the culture medium was lowered to room temperature, the cells would automatically detach from the culture dish in the form of single-layer films, and the cell sheets could be harvested. The temperature-sensitive culture dish was taken out from the incubator, the culture medium was removed, and the sheet-forming culture medium pre-cooled at 4° C. was added. After 0.25-1 hour, it was observed that the cell sheets began to detach from the edge of the culture dish. FIG. 2 shows a photo of the harvested cell sheet. It can be seen that the sheet is gray-white, with a dense structure and a smooth surface.

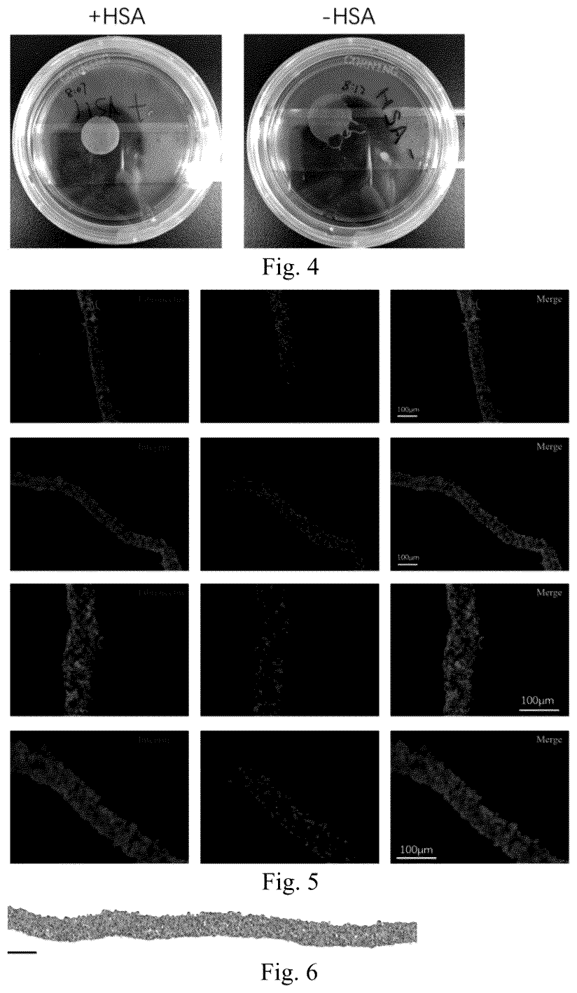

The results showed that coating human fibrinogen on a temperature-sensitive culture dish in advance can promote cell adhesion and sheet formation. When the coating matrix was not used for coating, the cell sheet detached in advance and the sheet was in a poor condition (FIG. 3). Cell-to-cell connections can be promoted by adding human serum albumin to the sheet-forming medium. When inoculated at a high cell amount and without adding human serum albumin to the sheet-forming medium, although the sheet morphology can be maintained, the connections between cells are relatively loose and the edges of the sheet are broken (FIG. 4).

The mesenchymal stem cell sheet obtained above was fixed with 4% paraformaldehyde, and then the cell sheet was prepared into tissue sections by the frozen section method for immunofluorescence staining (FIG. 5). The steps for sectioning and staining were as follows: the fixed mesenchymal stem cell sheets were taken out and put into an OCT embedding agent for embedding, and then a freezing microtome was used to cut the sheets into 10 μm thick sections. Sections were stained with monoclonal antibodies to fibronectin or integrin-β1, incubated overnight, and then stained with FITC-labeled fluorescent secondary antibodies. After staining was completed, a mounting medium containing Hoechst 33258 was added dropwise to cover the tissue sections. Finally, the tissue sections were covered with coverslips and observed under a fluorescence microscope.

By paraffin embedding and sectioning, the cell sheets were made into tissue sections for H&E staining (FIG. 6). The steps for sectioning and staining were as follows: the fixed cell sheets were dehydrated and then embedded in paraffin. Then the sheets were cut into 4-7 μm thick sections with a microtome to obtain paraffin sections. Then the sections were placed sequentially in xylene I for 10 min-xylene II for 10 min-absolute ethanol I for 5 min-absolute ethanol II for 5 min-95% alcohol for 5 min-90% alcohol for 5 min-80% alcohol for 5 min-70% alcohol for 5 min- and washed with distilled water. The sections were stained with hematoxylin for 3-8 minutes, washed with tap water, treated with 1% hydrochloric acid alcohol for a few seconds, rinsed with tap water, returned to blue with 0.6% ammonia, and rinsed with running water. The sections were stained in an eosin staining solution for 1-3 minutes. The sections were placed sequentially into 95% alcohol I for 5 min-95% alcohol II for 5 min-absolute ethanol I for 5 min-absolute ethanol II for 5 min, xylene I for 5 min-xylene II for 5 min to dehydrate and make them transparent. The sections were taken out of xylene to dry slightly, and sealed with neutral gum. The sections were observed under a microscope and photos were taken.

The results show that the cell sheets are composed of simple cells and extracellular matrix components secreted by the cells, with a thickness of about 10 cells stacked. The extracellular matrix components can be stained by fibronectin and integrin-β1.

### Example 3. Characteristics of Cell Sheets

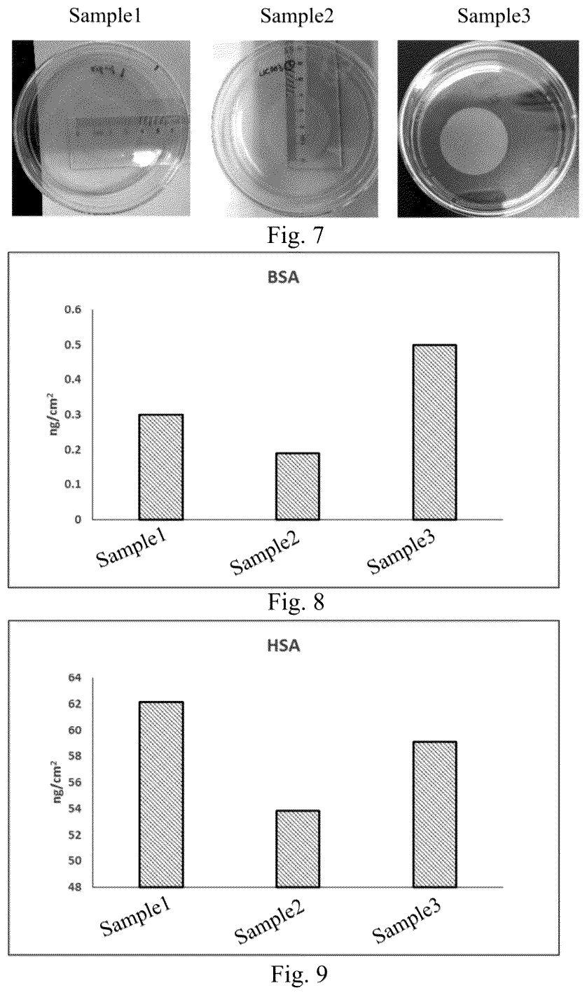

For the three batches of sheets prepared (sample 1, sample 2, and sample 3) (FIG. 7), the cell viability, total cell volume, and cell apoptosis were tested.

The steps for the apoptosis test were as follows: the umbilical cord mesenchymal stem cell sheets were digested into single cells and resuspended to a cell concentration of 2˜5×10⁵/ml. Then the Annexin V/PI apoptosis staining kit was used for staining according to the instructions. The steps were as follows: 1. washing the cells twice with Binding Buffer; 2. resuspending the cells with 195 μL Binding Buffer and adding 5 μL Annexin V staining solution. Incubating the cells in the dark for 30 minutes. 3. washing the cells twice with Binding Buffer and resuspending the cells in 190 μL Binding Buffer. Adding 10 μL PI staining solution. 4. Testing the cells after staining with flow cytometry. Those cells that are negative for Annexin V/PI staining are normal cells without apoptotic.

In addition, the residues of substances introduced during the preparation of cell sheets were tested.

### 1. Test of Bovine Serum Albumin Residual

The mesenchymal stem cell sheets were removed from the protective solution, and digested with 1 mL TryPLE at 37° C. for 3 minutes. During the digestion process, 50 μL of 20% human serum albumin injection was added to ensure that bovine serum albumin was not destroyed during the digestion process. Then 1 mL of sheet-forming medium was added to terminate the digestion, and the sheets were pipetted into single cells with a 1 mL pipette, and centrifuged at 300 g for 5 minutes. Afterwards, the supernatant was taken, and the content of bovine serum albumin was tested with an anti-bovine serum albumin antibody by ELISA. See FIG. 8 for the results. FIG. 8 shows that the residual bovine serum albumin content in the sheets is less than 0.5 ng/cm².

### 2. Human Serum Albumin Residual Detection

The mesenchymal stem cell sheets were moved from the protective solution, digested with 3 mL TryPLE at 37° C. for 3 minutes, then the digestion was terminated with 7 mL PBS. The sheets were pipetted into single cells with a 1 mL pipette, and centrifuged at 300 g for 5 minutes. Afterwards, the supernatant was taken and the content of human serum albumin was tested with anti-human serum albumin HSA antibody by ELISA. See FIG. 9 for the results. FIG. 9 shows that the residual human serum albumin content in the sheets is 53-62 ng/cm².

### 3. Human Fibrinogen Residual Detection

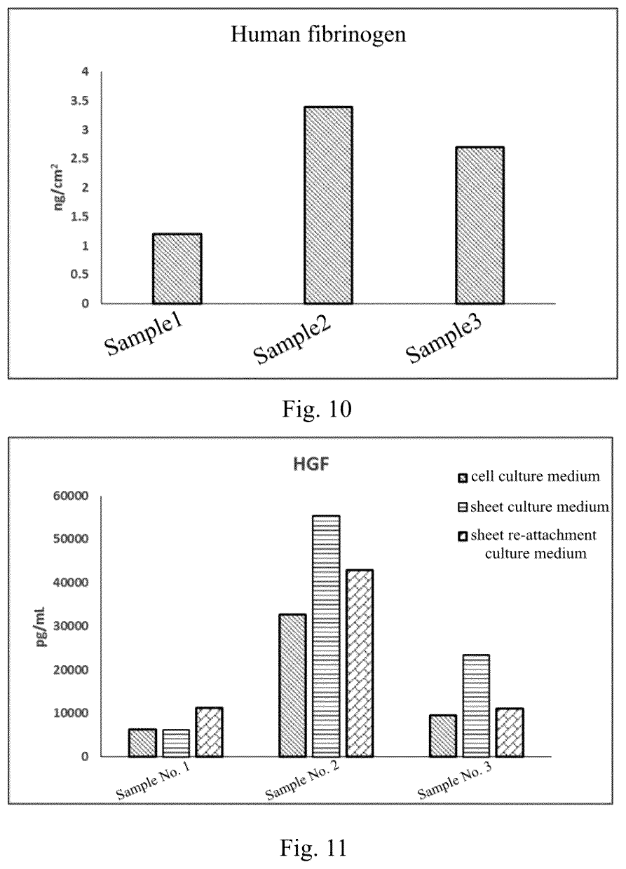

The mesenchymal stem cell sheets were removed from the protective solution, lysed with 5 mL of lysis solution (physiological saline+0.5% Triton+2.5% protease inhibitor) at 4° C. for 20 minutes, centrifuged at 12000 g at 4° C. for 20 minutes. Afterwards, the supernatant was taken and the content of human fibrinogen was tested with an anti-human fibrinogen antibody by ELISA. See FIG. 10 for the results. FIG. 10 shows that the residual human fibrinogen content in the sheets is 1.2-3.4 ng/cm².

### Example 4. Functional Tests of Cell Sheets

### 1. Test of Cytokines Secreted by the Cell Sheets

The cell culture medium was obtained as follows: replacing the cell culture medium in culture after subculture and before preparing the sheets with fresh medium (α-MEM supplemented with 20 IU/mL bFGF and 10% FBS), culturing for 24 hours at 37° C. with 5% carbon dioxide.

The sheet culture medium was the culture medium without cells obtained after sheet formation.

The sheet re-attachment culture medium was obtained as follows: washing the obtained sheet (e.g. 3 times with PBS), attaching it to the culture container, and adding umbilical cord mesenchymal stem cell culture medium (supplemented with 10% FBS and 20 IU/mL bFGF α-MEM), culturing for 24 hours at 37° C. with 5% carbon dioxide.

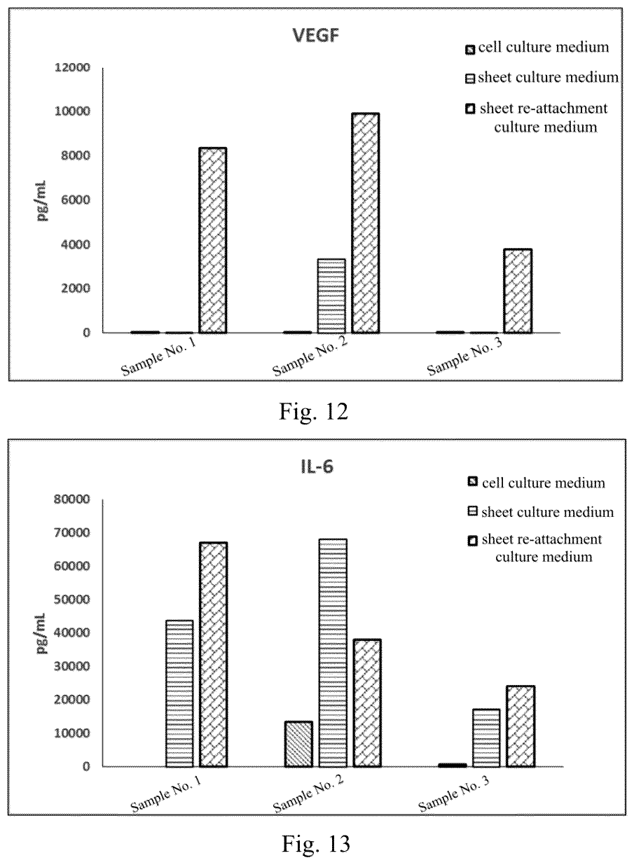

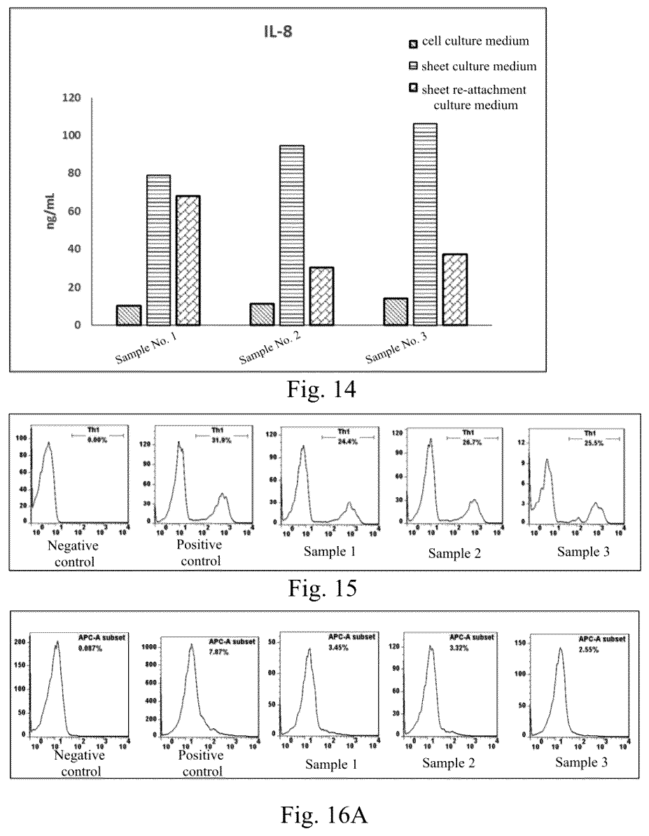

The cytokine content in the cell culture medium, sheet culture medium and sheet re-attachment culture medium were tested according to the instructions of the enzyme-linked immunoassay kit. The cytokines tested were hepatocyte growth factor (HGF) (see FIG. 11), vascular endothelial growth factor (VEGF) (see FIG. 12), interleukin-6 (IL-6) (see FIG. 13) and interleukin-8 (IL-8) (see FIG. 14).

The above results show that after preservation of the sheet, the ability to secrete the cytokine HGF was not decreased, and the ability of the cell sheet to secrete the cytokine HGF was stronger than that of the cells; after preservation of the sheet, the ability to secrete the cytokine VEGF was not decreased, and the ability to secrete the cytokine VEGF was stronger than that of cells.

### 2. The Inhibitory Effect of the Sheet on the Th1 Lymphocyte Subpopulation

The obtained sheet was washed three times with PBS, attached to a culture container and then co-cultured with lymphocytes. An experimental group (the sheet+lymphocyte+lymphocyte activator), a negative control group (the sheet+lymphocyte) and a positive control group (lymphocyte+lymphocyte activator) were set up, and the inhibitory effect of the sheet on Th1 lymphocyte subpopulation was tested by flow cytometry. See FIG. 15 for the results. FIG. 15 shows that the sheet has an inhibitory effect on lymphocyte subtype Th1 cells in an inflammatory environment, indicating that the sheet has immunomodulatory and anti-inflammatory effects.

### 3. The Inhibitory Effect of the Sheet on Lymphocyte Proliferation

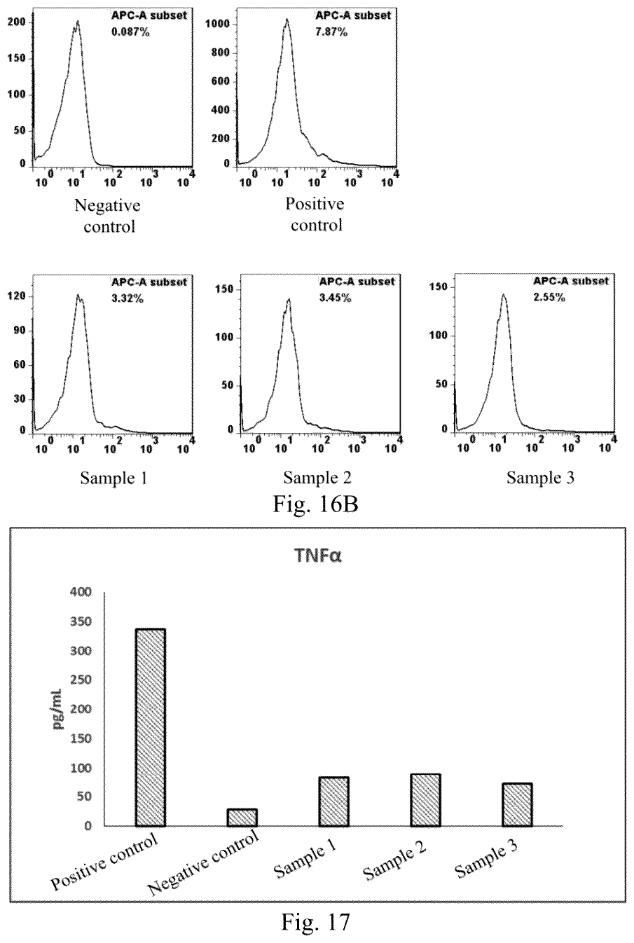

The obtained sheet was washed three times with PBS, attached to a culture container and then co-cultured with lymphocytes. An experimental group (the sheet+lymphocyte+lymphocyte stimulating agent), a negative control group (the sheet+lymphocyte) and a positive control group (lymphocyte+lymphocyte stimulating agent) were set up, the lymphocytes were labeled with BrdU and the inhibitory effect of the sheet on BrdU-positive lymphocyte population was tested by flow cytometry. See FIGS. 16A-16B for the results. FIGS. 16A-16B show that the sheet has an inhibitory effect on lymphocyte proliferation in an inflammatory environment, indicating that the sheet has immunomodulatory and anti-inflammatory effects.

### 4. Inhibitory Effect of the Sheet on TNFα Secretion by Lymphocytes

The obtained sheet was washed three times with PBS, attached to a culture container and then co-cultured with lymphocytes. An experimental group (the sheet+lymphocyte+lymphocyte stimulating agent), a negative control group (the sheet+lymphocyte) and a positive control group (lymphocyte+lymphocyte stimulating agent) were set up. The culture supernatant was collected, and the secretion of TNFα in each group was tested with anti-TNFα antibody by ELISA method, thereby calculating the inhibitory effect of the sheet on the TNFα secretion by lymphocytes. See FIG. 17 for the results. FIG. 17 shows that the sheet has an inhibitory effect on the secretion of TNFα by lymphocytes in an inflammatory environment, indicating that the sheets have immunomodulatory and anti-inflammatory effects.

### 5. Flow Cytometric Tests of Surface Markers

The umbilical cord mesenchymal stem cell sheets were digested into single cells and resuspended to a cell concentration of 1×10⁶/ml. 200 μL of the cells were taken into the flow tube, and the corresponding CD73, CD90, CD105, CD11b, CD19, CD34, CD45, and HLA-DR fluorescent staining antibodies were added to each tube according to the instructions. After incubation in the dark for 30 minutes, the cells were washed twice with PBS buffer solution, and then the stained cells were tested using a flow cytometer.

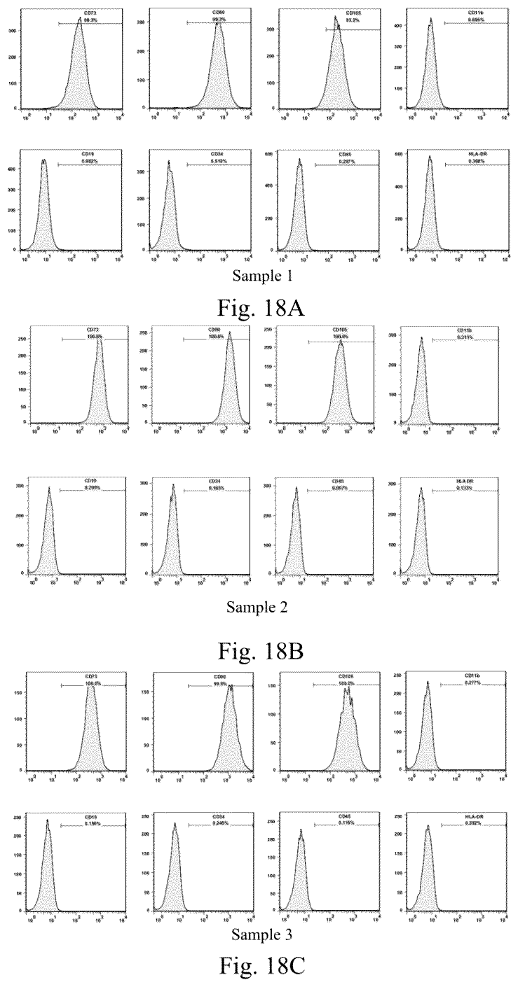

The flow cytometry results of cell sheet surface markers are shown in FIG. 18A-18C. The cell surface markers in the cell sheets all meet the requirements (CD73, CD90, CD105 positive rate>95%, CD11b, CD19, CD34, CD45, HLA-DR negative rate<2%). It shows that the cells contained in the cell sheets prepared by the present disclosure have stem cell-specific surface markers.

6. The Cells in the Sheets have the Ability of Three-Direction Differentiation by Induction

The umbilical cord mesenchymal stem cell sheets were digested into single cells, and inoculated into a suitable culture vessel according to the ratio in the reagent instructions for the three-direction differentiation by induction. When the cells for osteogenic induction testing grew to about 70% confluence, and the cells for adipogenic induction testing grew to more than 90% confluence, osteogenic and adipogenic induction mediums were added respectively. During chondrogenic induction, a certain number of cells were centrifuged to the bottom of the centrifuge tube, and then a chondrogenic induction medium was added. After the cells were aggregated into pellets, the cell pellets were kept away from the bottom of the tube to ensure complete contact with the induction medium. All cells were tested after induction and culture for more than 21 days. Osteogenesis induction can be stained with Alizarin Red, etc., adipogenesis induction can be stained with Oil Red O, etc., and chondrogenesis induction can be stained with Alcian Blue, etc.

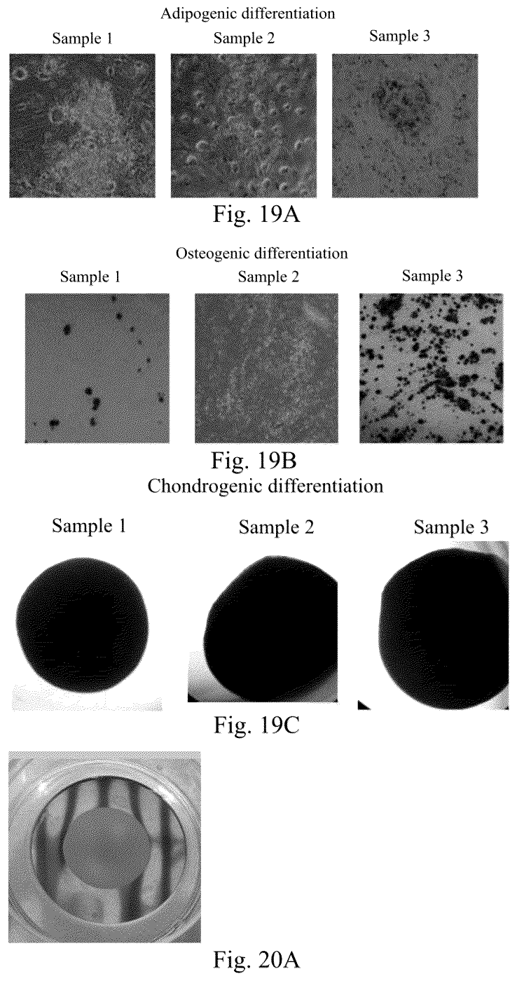

The results of the three-direction differentiation by induction are shown in FIGS. 19A-19C. It can be seen that the cells contained in the cell sheets can achieve adipogenic, osteogenic, and chondrogenic differentiation before and after preservation, and the staining was positive. It shows that the cell sheets prepared by the present disclosure have good differentiation potentials.

### Example 5. Other Coating Matrixes and Sheet-Forming Medium

The cell sheets were prepared with different combinations of coating matrix and sheet-forming medium according to the preparation process as described above. The results are as follows in Table 1.

TABLE 1
Cell sheets prepared with different combinations
of coating matrix and sheet-forming medium

| | Medium | | | | |
|---|---|---|---|---|---|
| | | | | DMEM + | BI XF + |
| | DMEM + | | | BI | BI |
| Coating | 10% | DMEM + | | Blood | Blood |
| Matrix | Serum | HSA 1% | Yocon | Substitute | Substitute |
| 100% Serum | Sheet | n/a | n/a | n/a | n/a |
| | formation | | | | |
| 20% HSA | n/a | No sheet | No sheet | n/a | n/a |
| | | formation | formation | | |
| Gelatin | n/a | Sheet | Sheet | No sheet | No sheet |
| | | formation | formation | formation | formation |
| PDL | n/a | Sheet | n/a | n/a | n/a |
| | | formation | | | |
| Vitronec- | n/a | Sheet | n/a | n/a | n/a |
| tinXF | | formation | | | |
| Fibrinogen | n/a | Sheet | n/a | n/a | n/a |
| | | formation | | | |
| Recombinant | n/a | Sheet | n/a | n/a | n/a |
| Fibronectin | | formation | | | |

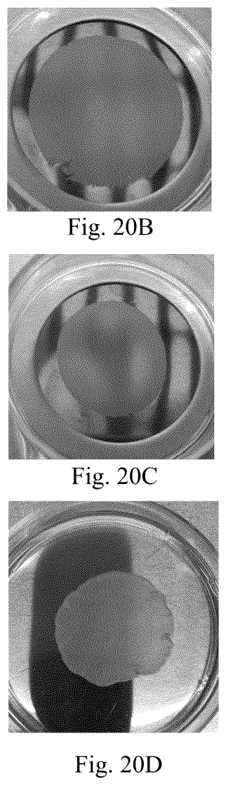

FIGS. 20A-20D show photographs of sheets prepared from gelatin (concentration 0.1% w/w) coating+HSA-containing sheet-forming medium (FIG. 20A), vitronectin XF (concentration 10 μg/mL) coating+HSA-containing sheet-forming medium, PDL (concentration 0.1 mg/mL) coating+HSA-containing sheet-forming medium (FIG. 20C) and recombinant fibronectin (concentration 10 μg/mL) coating+HSA-containing sheet-forming medium respectively. Gelatin, vitronectin XF and PDL have lower costs and are conducive to large-scale production and application.

1. A method of preparing a cell sheet, the method comprising the following steps:
a. cultivating and passaging cells;
b. transferring the cells to a temperature-sensitive culture dish coated with a matrix and culturing the cells in a sheet-forming medium to form a sheet in the culture dish, wherein the sheet-forming culture contains a basal culture medium and human serum albumin and does not contain animal-derived components and exogenous growth factors; and
c. detaching the cells from the temperature-sensitive culture dish by lowering the temperature.

2. The method of claim 1, wherein the cell sheet is a stem cell sheet, such as a mesenchymal stem cell sheet.

3. The method of claim 1, wherein the matrix is selected from the group consisting of fibronectin, laminin, gelatin, collagen, vitronectin and human fibrinogen, e.g., wherein the matrix is human fibrinogen, optionally wherein the concentration of the human fibrinogen is 0.1-10 mg/mL, such as 0.2-5 mg/mL, such as 1-2.5 mg/mL.

4. The method of claim 1, wherein the matrix is poly-D-lysine (PDL).

5-6. (canceled)

7. The method of claim 1, wherein in step b, the basal medium in the sheet-forming medium is selected from the group consisting of DMEM (high glucose), DMEM (low glucose), RPMI1640, α-MEM, DMEM/F12 and F12, preferably α-MEM.

8. The method of claim 1, wherein in step b, the concentration of the human serum albumin in the sheet-forming medium is 0.1-10%, such as 0.1-5%, such as 0.5-2%.

9. The method of claim 1, wherein in step b, the sheet-forming culture medium further comprises:
(1) glycine, L-alanine, L-aspartic acid, L-asparagine, L-glutamic acid, L-proline and L-serine, and/or
(2) L-Glutamine, optionally wherein the concentration of the L-glutamine is 0.5 mM to 4 mM, preferably 2 mM,
optionally, wherein the concentration of the glycine, the L-alanine, the L-aspartic acid, the L-asparagine, the L-glutamic acid, the L-proline and the L-serine each is 50 μM to 200 μM, preferably 100 μM.

10-11. (canceled)

12. The method of claim 1, wherein the medium used for culture and passage in step a is a serum-containing medium or a serum-free medium supplemented with one or more exogenous growth factors.

13. The method of claim 12, wherein the serum-free medium is selected from the group consisting of: RPMI1640, DMEM, α-MEM, DMEM/F12 and F12 serum-free medium, and the exogenous growth factor is selected from one or more of: vitamin C, sodium selenate, hydrocortisone, insulin, transferrin, human serum albumin, progesterone, putrescine, biotin, sodium pyruvate, ethanolamine, carnitine, amino acids, vitamins, glutathione, linoleic acid and linolenic acid.

14. The method of claim 12, wherein the serum-free medium is a commercial medium selected from the group consisting of: CTS™ Stem Pro™ MSC SFM, MesenCult™-ACF medium, MesenCult™-ACF Plus medium, and MesenCult™-XF Medium.

15. The method of claim 1, wherein the method further comprises a step of washing the cells after step a and before step b.

16. The method of claim 2, wherein the mesenchymal stem cells are derived from a tissue selected from the group consisting of: amniotic fluid, amniotic, chorion, chorionic villi, decidua, placenta, umbilical cord blood, Wharton's jelly, umbilical cord, adult bone marrow, adult peripheral blood, and adult adipose tissue.

17. The method of claim 2, wherein the mesenchymal stem cells is selected from the group consisting of umbilical cord mesenchymal stem cells, placental mesenchymal stem cells, adipose mesenchymal stem cells and bone marrow mesenchymal stem cells.

18. The method of claim 17, wherein the mesenchymal stem cells are umbilical cord mesenchymal stem cells, and wherein the method further comprises a step of obtaining mesenchymal stem cells from the umbilical cord before step a).

19. The method of claim 18, wherein obtaining umbilical cord mesenchymal stem cells from the umbilical cord comprises the steps of:
i. separating a Wharton's jelly from the umbilical cord;
ii. cutting the Wharton's jelly into small tissue pieces, and culturing the tissue pieces in a serum-free culture system for a sufficient time to allow the mesenchymal stem cells to migrate from the tissue pieces; and
iii. removing the tissue pieces to obtain the umbilical cord mesenchymal stem cells when the mesenchymal stem cells grow to 50%-100% confluence, such as 70%-100% confluence or 80%-100% confluence.

20. The method of claim 1, wherein in step b, the sheet-forming culture temperature is about 37° C., and the cells are detached from the temperature-sensitive culture dish by reducing the temperature to 4-32° C.

21. A cell sheet prepared by the method of claim 1.

22. The cell sheet of claim 21, wherein the residual amount of bovine serum albumin in the cell sheet is ≤1 ng/cm², the residual amount of human serum albumin is 10 ng/cm²-15000 ng/cm², and the residual amount of human fibrinogen is 0.5 ng/cm²-5 ng/cm².

23. Use of the cell sheet prepared by the method of claim 1 in the manufacture of a medicament for modulating an inflammatory response or treating an autoimmune system disease in a subject.

24. Use of the cell sheet prepared by the method of claim 1 in the manufacture of a medicament for damage repair of damaged tissue in a subject.
